(19) United States
(12) Patent Application Publication
(10) Pub. No.: US 2013/0310713 A1
(43) Pub. Date: Nov. 21, 2013

(54) DEVICE FOR MEASURING COMPLIANCE WITH OXYGEN THERAPY USING A THREE-DIMENSIONAL ACCELEROMETER

(71) Applicants: Claude Weber, Milly la Foret (FR); Philippe Bernard, Goincourt (FR)

(72) Inventors: Claude Weber, Milly la Foret (FR); Philippe Bernard, Goincourt (FR)

(21) Appl. No.: 13/983,217

(22) PCT Filed: Jan. 11, 2012
(86) PCT No.: PCT/FR2012/050071

§ 371 Date: Aug. 1, 2013

(30) Foreign Application Priority Data
Feb. 2, 2011 (FR) 1150798

(51) Int. Cl. A61B 5/00; A61B 5/11; A61M 16/00

(52) U.S. Cl. CPC A61B 5/4833; A61M 16/00; A61B 5/0002; A61B 5/1118
USPC 600/595

(57) ABSTRACT

The invention relates to a device for measuring patient compliance with oxygen therapy, including a casing (4) comprising means for detection of the oxygen therapy in order to evaluate data relating to the treatment; and means for measuring a state of physical activity of the patient. Preferably, the means for measuring a state of physical activity of the patient comprise at least one sensor which senses movement of the patient's body and outputs data relating to the spatial displacement of the patient's body, for example a three-dimensional accelerometer (28); and processing means (40, 42) for processing the body displacement data in order to supply a state of physical activity of the patient chosen from a sleeping state, a resting state and an active state.

### CROSS REFERENCE TO RELATED APPLICATIONS

This application is a §371 of International PCT Application No. PCT/FR2012/050071, filed Jan. 11, 2012, which claims priority to French Application 1150798, filed Feb. 2, 2011, the entire contents of which are incorporated herein by reference.

### BACKGROUND

The present invention relates to a device for measuring compliance with oxygen therapy treatment, and a corresponding measurement system and method.

More particularly, the present invention relates to the field of compliance with medical oxygen therapy treatment, in particular in home care situations.

Generally, knowing the compliance by a patient with a treatment, that is to say measuring the actual time for which he or she follows his or her treatment, is essential, because it provides a way of knowing the effectiveness of the treatment and thus of adjusting the prescription for the treatment by the treating doctor.

Compliance monitoring is vitally important in the case of oxygen therapy treatment, generally prescribed for patients afflicted with breathing inadequacies, in particular chronic obstructive bronchopneumopathy, called “COBP”.

In practice, oxygen therapy treatment that is correctly followed can lead to a significant improvement in the quality of life of the patient and reduce hospitalizations.

However, the effects of oxygen therapy are negligible, even nonexistent, if the patient does not comply with his or her treatment, for example if the taking of oxygen is performed for a duration less than fifteen hours per day.

The document WO-A-2009/136101 describes a device intended for the implementation of a system making it possible to detect, in a patient, compliance with oxygen therapy treatment with added oxygen. This device comprises means for detecting the oxygen therapy treatment to evaluate data relating to the treatment and means for recording and transmitting these data.

This remote monitoring device used by the patient in his or her home provides for a daily measurement of the time for which the patient has followed his or her treatment. It thus constitutes an aid to the treating doctor to predict aggravations to the sickness and reduce the number and duration of hospitalizations.

However, this device does not take into account the variability of the oxygen needs of the patient, which has a great influence on the effectiveness of the oxygen therapy treatment.

For its part, the document FR-A-2916291 proposes a universal compliance device that can be used in the context of oxygen therapy treatment comprising a patient data input device with flow rate and breathing activity sensor, data storage means and a communication interface allowing for communication with a remote data processing unit so as to be able to display information or print out reports. Optionally, this device may comprise a sensor for sensing the physical activity of the patient.

However, no information is given regarding said sensor sensing physical activity of the patient, or regarding the usefulness of the measurements that it is likely to perform.

Another compliance device that can be used in the context of oxygen therapy treatment is also taught by the document EP-A-1661595.

### SUMMARY

The present invention aims to propose an improved device for measuring compliance with oxygen therapy treatment which takes into account the variability of the patient's oxygen needs and thus makes it possible to more effectively evaluate the efficiency of the oxygen therapy treatment.

Furthermore, the present invention relates to a device for measuring compliance with oxygen therapy treatment by a patient, comprising a module incorporating means for detecting the oxygen therapy treatment, such as one or more sensors, to evaluate data relating to the treatment; and means for measuring a state of physical activity of the patient.

According to the invention, the means for measuring a state of physical activity of the patient comprise:
at least one sensor for sensing movements of a patient's body which outputs data relating to the spatial displacement of the patient's body; and
means for processing the body displacement data, such as one or more microprocessors, to provide a state of physical activity of the patient, in particular a state of physical activity of the patient chosen from a sleeping state, a resting state and an active state, or even any other state of physical activity.

It should be noted that a sleeping state does not necessarily indicate that the patient is sleeping. It may also correspond to a state of inactivity of the patient.

In practice, the inclusion of the state of physical activity of the patient by the device according to the invention, and more specifically by, in particular, the means for measuring a state of physical activity of the patient, makes it possible to correlate consumption of oxygen by the patient with this activity and thus more effectively evaluate the efficiency of the oxygen therapy treatment.

Furthermore, integrating these different functions in one and the same module, that is to say a single module, simplifies its use for the patient. Preferably, the module is portable and seal-tight.

Advantageously, the module of the device of the invention also comprises means for storing data relating to the treatment and to the state of physical activity of the patient; or means for transmitting the data relating to the treatment and to the state of physical activity of the patient to a remote server; or both.

Such a remote server may be situated in particular in the service provider center monitoring the patient. The treating doctor thus has real-time data on the compliance with the treatment followed by the patient, made available to him or her by the service provider.

Preferably, the storage means, such as one or more information storage memories, can also store data relating to the compliance measurement device.

Integrating all of the measurement, storage and data transmission means in a single module provides the patient and the treating staff with a device that is easy to use.

Advantageously, the body movement sensor comprises a three-dimensional accelerometer. Such a three-dimensional accelerometer offers the advantage of being able to reliably identify the displacements of the patient and being able to deduce therefrom his or her state from the abovementioned three states regardless of how it is positioned in relation to the body of the patient. It can thus be worn equally as a shoulder strap or at belt level, for example, without affecting the estimation of the type of activity of the patient.

The use of a single three-dimensional accelerometer is sufficient to reliably identify the state of the patient from the abovementioned three states. Obviously, if necessary, several accelerometers can be used.

Advantageously, the module is seal-tight. Thus, the patient can use it permanently even when taking a shower, which reinforces the relevance of the data recorded. In practice, patients suffering from COBP generally take their shower while being under oxygen therapy treatment, given that the input of oxygen is also recommended during this activity.

Moreover, the module is configured to be wearable, that is to say portable. Preferably, the weight of the module is less than 200 g and lies between 20 and 100 g, notably between 25 and 80 g, for example between approximately 30 and 50 g.

According to a preferred embodiment, the module comprises an integrated circuit card in which said oxygen therapy treatment detection means and said means for measuring a state of physical activity of the patient are provided. Preferably, said card comprises at least one microprocessor common to the oxygen therapy treatment detection means and to the means for measuring a state of physical activity of the patient.

Advantageously, the device comprises a single electrical power supply battery for the card. This battery allows the module to operate in total autonomy, at least for one year, even more preferably for two years.

Moreover, the microprocessor makes it possible to implement, on the one hand, an algorithm capable of converting pressure measurements into patient breathing frequencies and deduce therefrom the daily treatment duration and, on the other hand, an algorithm capable of deducing a state of physical activity of the patient from the body displacement data.

Furthermore, the module comprises at least one passage for circulating oxygen inside, said passage being insulated from the rest of the module, which means that it is seal-tight to the rest of the module.

Depending on the case, the device of the invention may comprise one or more of the following features:
the pressure sensor(s) and the accelerometer are connected to a first microprocessor which carries out a preprocessing of the measurement signals obtained from the pressure sensor(s) and from the accelerometer.
a second microprocessor determines, from the signals preprocessed by the first microprocessor, the presence or absence of oxygen therapy treatment and controls the recording of data in the information storage means.
the second microprocessor controls the recording of data chosen from the oxygen therapy treatment duration, the maximum breathing frequency and the average breathing frequency.
the second microprocessor also determines, from the indicators of the body movements of the patient, a state of activity of the patient and controls the recording of the state of activity of the patient in the information storage means.

The invention also relates to a system for measuring compliance with oxygen therapy treatment followed by a patient, comprising the measurement device according to the invention and means for transmitting information between the measurement device and a remote server.

By way of example, such information transmission means comprise an office or laptop computer, a personal digital assistant (PDA), a modem or other suitable information transmission device, via which the data relating to the treatment and the state of physical activity of the patient are transmitted to the remote server situated in a care center.

Advantageously, the measurement system also comprises a source of oxygen; and a cannula delivering oxygen to the patient's airways, said measurement device being configured to be connected to the oxygen source and to the cannula.

The invention also relates to a method for measuring compliance with oxygen therapy treatment by a patient, comprising the following steps:
detection of the oxygen therapy treatment to evaluate data relating to the treatment; and
measurement of a state of physical activity of the patient.

Advantageously, the step of measurement of a state of physical activity of the patient comprises:
a step of measuring data relating to the spatial displacement of the patient's body; and
a step of processing the body displacement data to provide a state of physical activity of the patient chosen from a sleeping state, a resting state and an active state.

### BRIEF DESCRIPTION OF THE DRAWINGS

There now follows a description, more detailed but of a nonlimiting nature, of an exemplary embodiment of a device and a system according to the invention, with reference to the appended figures in which:

FIG. 1 is a block diagram illustrating the structure of a compliance measurement system according to one embodiment of the invention;

FIG. 2 illustrates the positioning of the axes of a three-dimensional accelerometer in the device of FIG. 3;

FIG. 3 is a block diagram illustrating the structure of a compliance measurement device according to one embodiment of the invention;

FIG. 4 is a diagram illustrating the electronic architecture of the device of FIGS. 2 and 3; and

FIG. 5 is a timing diagram illustrating the operation of the compliance measurement method according to one embodiment of the invention.

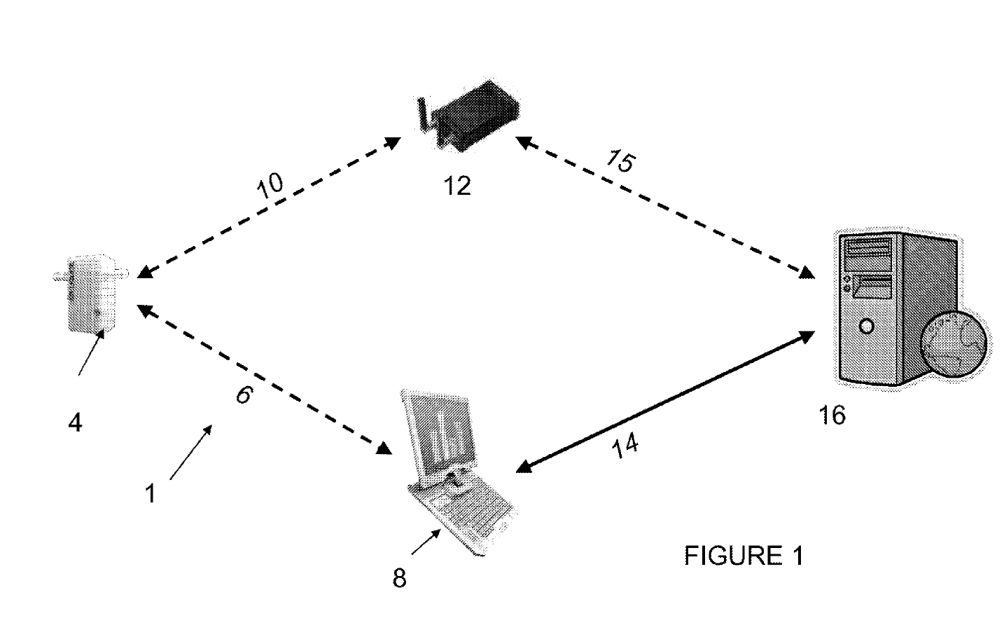

FIG. 1 illustrates a system 1 for measuring compliance with oxygen therapy treatment implementing a device according to the invention comprising a module 4.

### DESCRIPTION OF PREFERRED EMBODIMENTS

The module 4 is wearable or portable, that is to say configured to be worn by the patient in his or her home in particular, for example on the belt by virtue of a suitable attachment system or around the neck by virtue of a pendant attachment system.

The module 4 is connected, via an information transmission link 6, to recording means 8 suitable for recording data, for example a personal computer, a personal digital assistant (PDA) or similar.

The module 4 is also connected, via an information transmission link 10, to communication means 12 forming a data communication gateway, for example a GSM or GPRS modem.

The transmission links 6 and 10 are preferably radiofrequency (RF) links in the ISM (Industrial Scientific Medical) frequency bands located between 800 MHz and 5 GHz, preferably between 850 MHz and 3 GHz and even more preferably 868 MHz or 2.4 GHz.

Furthermore, the recording means 8 are connected via a data transmission network 14, such as the Internet, to a remote server 16 situated in the service provider center responsible for the patient.

The communication means 12 are also connected to the remote server 16 via an information transmission link 15, for example a GSM or GPRS link, when the communication means 12 comprise a GSM or GPRS modem.

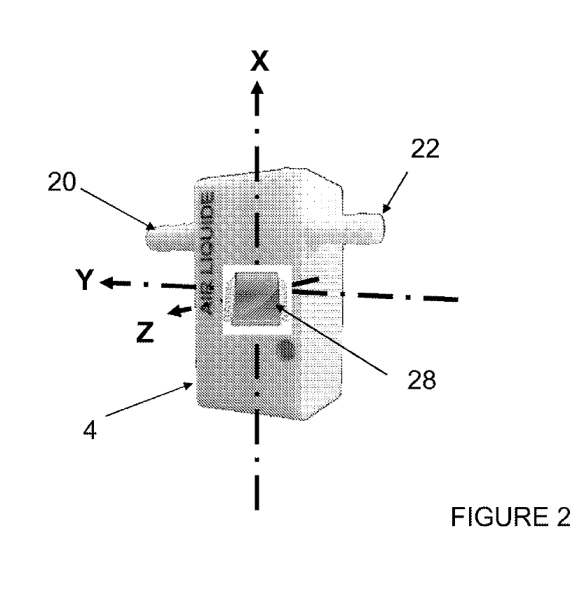

As appears in FIG. 2, the module 4 comprises, at the ends of the internal gas passage, two end fittings 20, 22 intended to connect it on the one hand to a source of oxygen (not represented) and on the other hand to a patient interface, for example a nasal cannula (not represented), or even a breathing mask.

An oxygen circulation passage is provided in the module 4, said passage being seal-tight in relation to the rest of the module, in particular the electronic components.

The oxygen source is chosen from the sources conventionally used in oxygen therapy, for example a compressed oxygen cylinder or an oxygen concentrator or even a liquid oxygen tank. Preferably, the oxygen flow rate is between 0.5 and 4 liters/minute. Obviously, this flow rate is matched to the medical prescription.

A standard cannula can be used, that is to say a cannula that is not specifically designed for use with the device of the invention. For example, it can be a cannula according to the standard NF EN 13544-2 (“Respiratory Therapy Equipment—Part 2: Tubing and Connectors”) having a connection end fitting, such as, for example, an Intersurgical™, Salter Labs™ or Octurno Medizintechnick™ cannula.

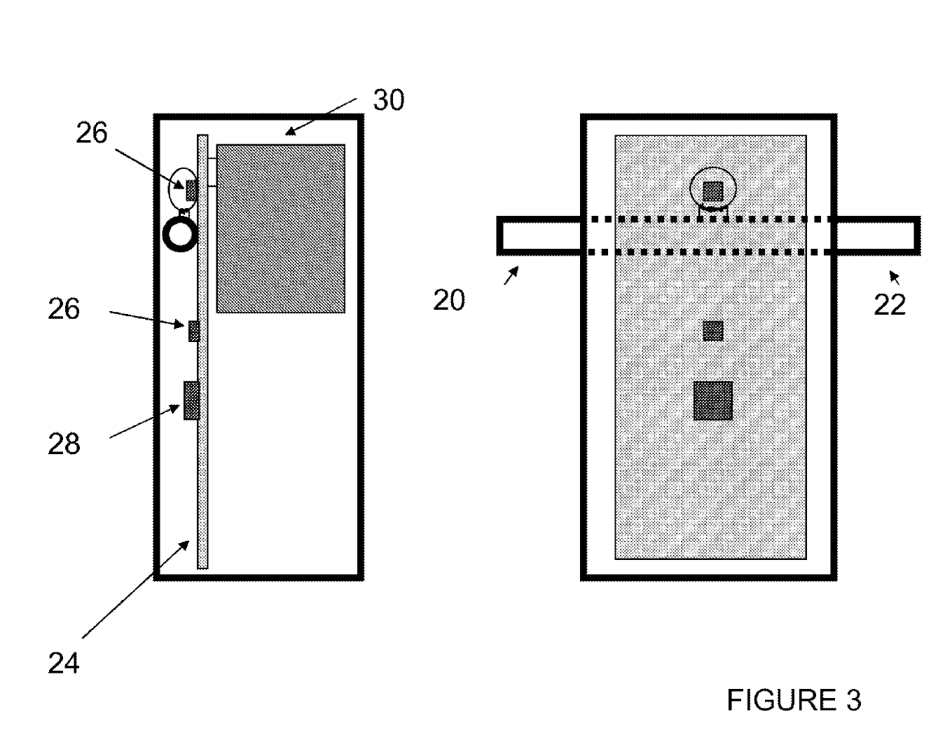

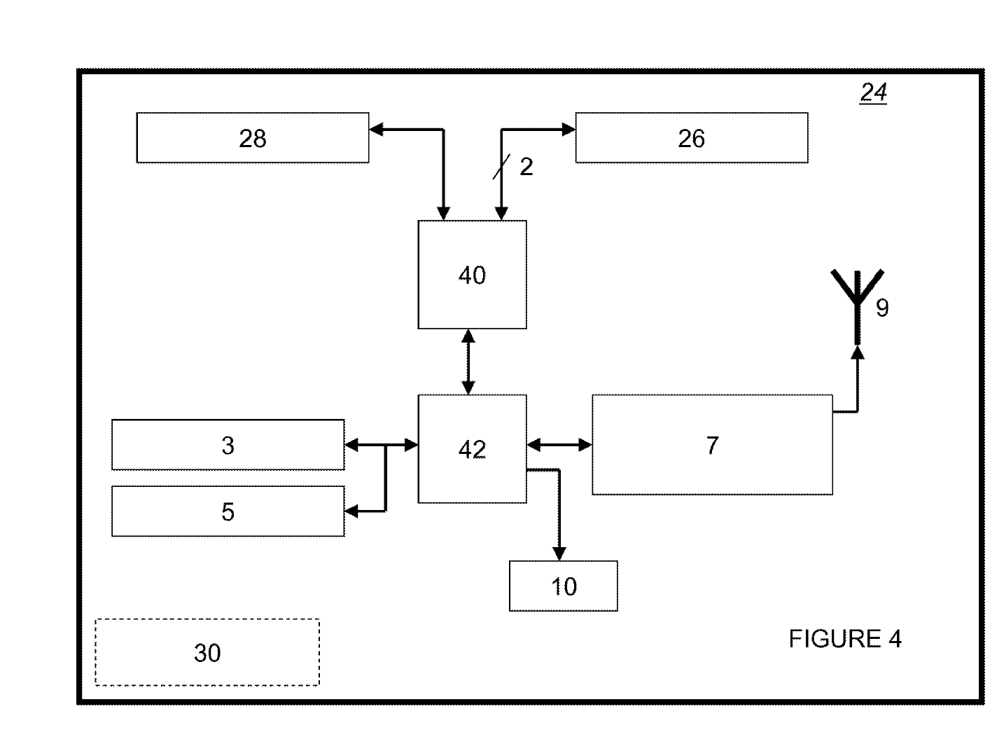

The module 4 also comprises an integrated circuit card 24. As schematically represented in FIGS. 3 and 4, this card 24 comprises two pressure sensors 26. The first sensor measures the pressure in the cannula due to the oxygen flow rate and to the variations linked to the inhalations (corresponding to a depression) and exhalations (corresponding to an overpressure) of the patient in the cannula, and the second sensor measures the atmospheric pressure. The card 24 is positioned for this purpose facing the oxygen circulation passage in the gas passage of the module 4.

The card 24 also comprises a three-dimensional accelerometer 28 with its three axes X, Y, Z oriented in relation to the card 24 as represented in FIG. 2.

The use of a three-dimensional accelerometer 28 makes it possible to have accurate body movement measurements regardless of the position of the module 4. Thus, the patient is free to wear this module 4 as he or she likes without affecting the estimation of the type of activity of the patient.

The module 4 also comprises an electrical power supply battery 30 for the card 24. This battery 30 has an autonomy of at least 1 year and if possible at least 2 to 3 years.

The pressure sensors 26 and the accelerometer 28 are connected to a microprocessor 40 which carries out a preprocessing of the measurement signals obtained from the pressure sensors 26 and from the accelerometer 28. For this, a first algorithm is implemented to transform the pressure values into patient breathing frequencies and a second algorithm is implemented that is capable of deducing indicators of the body movements of the patient from the measured accelerations.

A second microprocessor 42 determines, from the breathing frequencies of the patient, the presence or absence of oxygen therapy treatment and controls the recording of the oxygen therapy treatment duration as well as the maximum breathing frequency and the average breathing frequency in information or data storage means, comprising a FRAM memory 3 and a FLASH memory 5.

The second microprocessor 42 also determines, from the body movement indicators of the patient, a state of activity of the patient chosen from a sleeping or inactive state, a resting state and an active state. It also controls the recording of the state of activity of the patient in the storage means comprising the FRAM memory 3 and the FLASH memory 5.

The data storage memories 3 and 5 have, for example, a capacity of 1 to 3 Mb, notably 2 Mb, and an autonomy of at least 1 year.

Furthermore, the second microprocessor 42 controls the transmission of the oxygen therapy treatment duration, the maximum breathing frequency, the average breathing frequency and the state of activity of the patient, via a radiofrequency communication module 7 connected to an antenna 9, to the remote server 16. The microprocessors 40, 42 and the memories 3, 5 are incorporated in the card 24.

Furthermore, the second microprocessor 42 is connected to a light-emitting diode (LED) 10 which constitutes a means of communication with the patient.

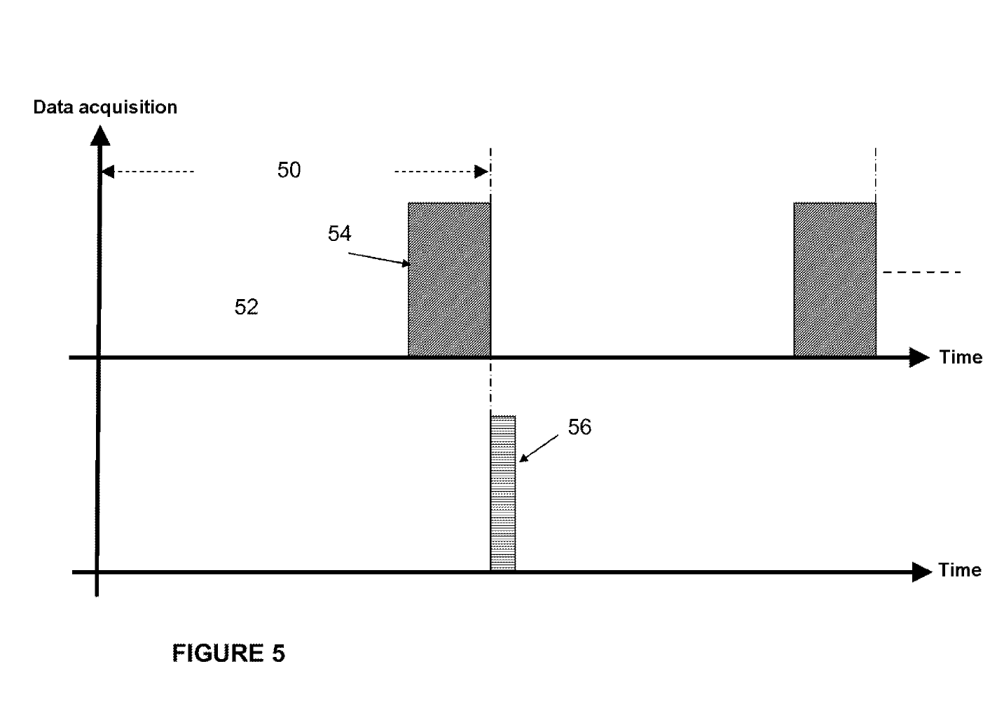

The timing diagram of FIG. 5 illustrates the various steps of the compliance measurement method of the invention.

In a step A, pressure and acceleration measurements are made during successive measurement cycles 50. Each measurement cycle 50 comprises a given measurement period 54, for example periods from several minutes to several hours, even several days, from the measurement sensors 26 and the accelerometer 28. The measurement period 54 is followed by a period 56 of processing of the results of these measurements by the microprocessor 40. During the period 52, no measurement is performed.

In a step B, the microprocessor 42 determines, during the period 56, located immediately after the cycle 50, the oxygen therapy treatment duration, the maximum breathing frequency, the average breathing frequency and the state of activity of the patient, and controls the storage of these data in the memories 44, 46.

The cycles 50 are repeated in time.

The device for measuring compliance with oxygen therapy treatment according to the invention suited to a use of home care type, based on monitoring the state of physical activity of the patient, namely sleeping state, resting state or active state, or even any other state of physical activity of the patient.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing i.e. anything else may be additionally included and remain within the scope of “comprising.” “Comprising” is defined herein as necessarily encompassing the more limited transitional terms “consisting essentially of” and “consisting of”; “comprising” may therefore be replaced by “consisting essentially of” or “consisting of” and remain within the expressly defined scope of “comprising”.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1.-15. (canceled)

16. A device for measuring compliance with oxygen therapy treatment by a patient, comprising a module (4) containing:
a system configured to detect the oxygen therapy treatment to evaluate data relating to the treatment; and
a system (28; 40, 42) configured to measure a state of physical activity of the patient, characterized in that the system configured to measure the state of physical activity of the patient comprises:
at least one sensor (28) for sensing a movement of a patients body which is adapted to output data relating to a spatial displacement of the patient's body; and
a data processing device (40, 42) configured to process the body spatial displacement data to provide the state of physical activity of the patient.

17. The device as claimed in claim 16, characterized in that the module (4) also comprises a data storage device (3, 5) configured to store data relating to the oxygen therapy treatment and to the state of physical activity of the patient.

18. The device as claimed in claim 17, characterized in that the module (4) also comprises a transmitter (7, 9) adapted for transmitting the data relating to the treatment and to the state of physical activity of the patient to a remote server (16).

19. The device as claimed in claim 16, characterized in that the body movement sensor comprises a three-dimensional accelerometer (28).

20. The device as claimed in claim 16, characterized in that the module (4) comprises an integrated circuit card (24) comprising said oxygen therapy treatment detection system and said system for measuring the state of physical activity of the patient.

21. The device as claimed in claim 20, characterized in that the device comprises a single power supply battery (30) for the integrated circuit card (24).

22. The device as claimed in claim 20, characterized in that the integrated circuit card (24) comprises at least one microprocessor (40, 42) common to the oxygen therapy treatment detection system and to the system for measuring the state of physical activity of the patient.

23. The device as claimed in claim 18, characterized in that at least one microprocessor (40, 42) is arranged in operable cooperation with the data storage device(3, 5) and the transmitter (7, 9).

24. The device as claimed in claim 16, characterized in that the module (4) comprises at least one passage adapted for circulating oxygen passing through said module (4), said passage being insulated from the rest of the module (4) and comprising two end fittings (20, 22) at its ends.

25. The device as claimed in claim 19, characterized in that the device also comprises one or more pressure sensors (26) and in that:
the pressure sensor(s) (26) and the three-dimensional accelerometer (28) are connected to a first microprocessor (40) which is configured to carry out a preprocessing of measurement signals obtained from the pressure sensor(s) (26) and from the three-dimensional accelerometer (28), and
a second microprocessor (42) which is configured to determine, from the signals preprocessed by the first microprocessor (40), a presence or an absence of oxygen therapy treatment and which is configured to control a recording of data in an information storage device(3, 5).

26. The device as claimed in claim 25, characterized in that the second microprocessor (42) is configured to control the recording of data chosen from an oxygen therapy treatment duration, a maximum breathing frequency and an average breathing frequency.

27. The device as claimed in claim 25, characterized in that the second microprocessor (42) also is configured to determine, from the spatial displacement of the patient's body, the state of activity of the patient and is configured to control the recording of the state of activity of the patient in the information storage means (3, 5).

28. The device as claimed in claim 16, characterized in that the data processing device (40, 42) configured to process the body spatial displacement data is configured to provide the state of physical activity of the patient chosen from a sleeping state, a resting state and an active state.

29. The device as claimed in claim 16, further comprising an oxygen source in fluid communication with an interface delivering the oxygen.

30. A method for measuring compliance with oxygen therapy treatment by a patient, comprising the following steps:
a) detecting of the oxygen therapy treatment in order to evaluate data relating to the oxygen therapy treatment; and
b) measuring of a state of physical activity of the patient characterized in that the step of measuring of the state of physical activity of the patient comprises:
a step (A) of measuring data relating to the spatial displacement of the patient's body; and
a step (B) of processing the body spatial displacement data to provide the state of physical activity of the patient chosen from a sleeping state, a resting state and an active state.
